(19) United States
(12) Patent Application Publication
(10) Pub. No.: US 2004/0049057 A1
(43) Pub. Date: Mar. 11, 2004

(54) Dihydro-furan-2-one derivatives, their intermediates and methods of manufacture

(75) Inventors: Frank J. Urban, Old Saybrook, CT (US); V. John Jasys, Griswold, CT (US); Zhengong B. Li, East Lyme, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/431,278

(22) Filed: May 7, 2003

Related U.S. Application Data
(60) Provisional application No. 60/380,678, filed on May 14, 2002.

(51) Int. Cl. C07D 307/93; C07D 305/12; C 07D 4 7/02

(52) U.S. Cl. 549/312

(57) ABSTRACT

This invention relates to dihydro-furan-2-one derivatives, their intermediates and methods of manufacture. As such, the present invention includes methods of making a compound of the formula (V-1)

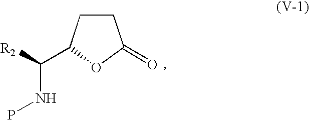

wherein R₂ and P are herein defined. The present invention also relates to the compounds used in such processes, as well as the compounds made by the processes.

### CROSS-REFERENCE TO RELATED APPLICATION

This application claims the benefit of priority of provisional Patent Application Serial No. 60/380,678 filed May 14, 2002, which is incorporated herein by reference in its entirety for all purposes.

### FIELD OF THE INVENTION

This invention relates to dihydro-furan-2-one derivatives, their intermediates, and methods of manufacture.

### BACKGROUND

Dihydroxyhexanoic acid derivatives are potent and selective inhibitors of MIP-1α, binding to the receptor CCR1 found on inflammatory and immunomodulatory cells (preferably leukocytes and lymphocytes). The CCR1 receptor is also sometimes referred to as the CC-CKR1 receptor. These compounds also inhibit MIP-1α (and the related chemokine shown to interact with CCR1 (e.g., RANTES and MCP-3)) induced chemotaxis of THP-1 cells and human leukocytes and are potentially useful for the treatment or prevention of autoimmune disea23017ses (such as rheumatoid arthritis, type I diabetes (recent onset), inflammatory bowel disease, optic neuritis, psoriasis, multiple sclerosis, polymyalgia rheumatica, uveitis, and vasculitis), acute and chronic inflammatory conditions (such as osteoarthritis, adult Respiratory Distress Syndrome, Respiratory Distress Syndrome of infancy, ischemia reperfusion injury, and glomerulonephritis), allergic conditions (such as asthma and atopic dermatitis), infection associated with inflammation (such as viral inflammation (including influenza and hepatitis) and Guillian-Barre), transplantation tissue rejection (chronic and acute), organ rejection (chronic and acute), atherosclerosis, restenosis, HIV infectivity (co-receptor usage), and granulomatous diseases (including sarcoidosis, leprosy and tuberculosis).

Dihydroxyhexanoic acid derivatives are described in co-pending U.S. patent application Ser. No. 09/380,269, filed Feb. 5, 1998 and 09/403,218, filed Jan. 18, 1999; and PCT publication numbers WO98/38167 and WO99/40061, commonly assigned to the assignee of the present invention and all of which are incorporated herein by reference in their entireties for all purposes.

### SUMMARY OF THE INVENTION

As embodied and broadly described herein, this invention, in one aspect, relates to methods of making compounds of the formula (V-1):

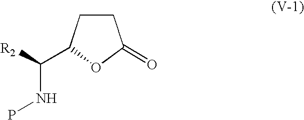

wherein:

P is a protecting group;

R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl(CH₂)ₘ—, (C₃-C₁₀)cycloalkyl-(CH₂)ₘ— or (C₂-C₉)heteroaryl-(CH₂)ₘ— groups may be optionally substituted with one, two, or three substituents independently selected from the group consisting of hydrogen, halogen, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-O(C═O)—, HO—(C═O)—(C₁-C₆)alkyl, (C₁-C₆)alkyl-O—(C═O)—(C₁-C₆)alkyl, (C₁-C₆)alkyl-(C═O)O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N(C═O)—, H₂N(C═O)—(C₁-C₆)alkyl, (C₁-C₆)alkyl-HN(C═O)—(C₁-C₆)alkyl, [(C₁-C₆)alkyl]₂N(C═O)—(C₁-C₆)alkyl, H(O═C)—NH—, (C₁-C₆)alkyl(C═O)—NH, (C₁-C₆)alkyl(C═O)[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and

m is 0, 1, 2, 3, or 4;

wherein the method comprises:

a) hydrolyzing a compound of the formula (VIg-1)

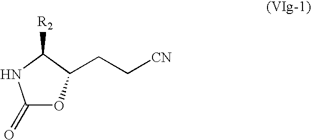

with an aqueous solution in the presence of a base,

b) protecting the amine group of the compound so formed, and

c) cyclizing the compound so formed with heat and an acid catalyst.

In the methods and compounds herein described, the variables shall have the same definition unless otherwise noted.

In one preferred embodiment, these methods further comprise formation of the compound of the formula (VIg-1) by reacting a compound of the formula (VIf-1)

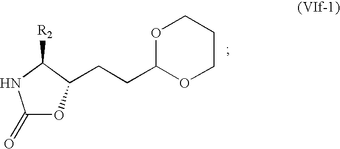

with hydroxylamine hydrochloride and an acid catalyst.

Moreover, a preferred embodiment also includes methods further comprising formation of the compound of the formula (VIf-1) by heating a compound of the formula (VIe-1)

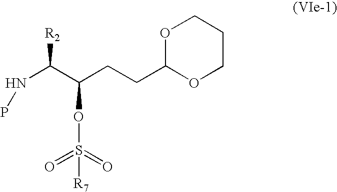

wherein R₇ is (C₁-C₆)alkyl or phenyl wherein the phenyl group may be optionally substituted with one, two, or three (C₁-C₆)alkyl, hydroxy, or halogen groups.

In another preferred embodiment, the methods further comprise formation of the compound of the formula (VIe-1) by reacting a compound of the formula (VId-1)

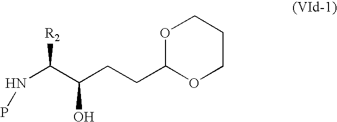

with a compound of the formula R₇—SO₂-halide in the presence of a base.

In yet another preferred embodiment, the methods further comprise formation of the compound of the formula (VId-1) by reducing a compound of the formula (VI-1)

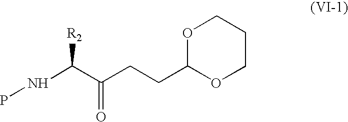

with a reducing agent.

In a second aspect, the present invention relates to methods of making compounds of the formula (V-1) wherein the method comprises heating a compound of the formula (VIb-1)

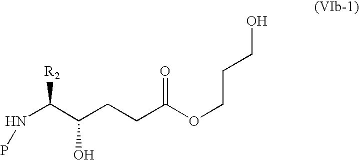

in the presence of an acid catalyst.

In one preferred embodiment, the methods further comprise formation of the compound of the formula (VIb-1) by reacting a compound of the formula (VIa-1)

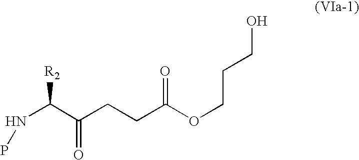

with a silylating agent and further reacting the compound so formed with a reducing agent.

In another preferred embodiment, the methods further comprise formation of the compound of the formula (VIa-1) by reacting a compound of the formula (VI-1)

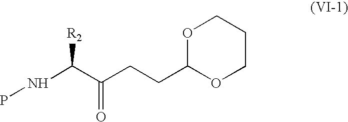

with ozone or hypochlorous acid.

In yet another preferred embodiment, the methods further comprise formation of the compound of the formula (VIa-1) by reacting a compound of the formula (VI-1)

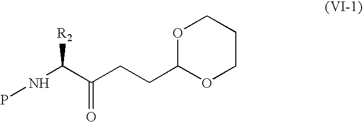

with hypochlorous acid and further reacting the compound so formed with hydrogen in the presence of a catalyst.

In a third aspect, the present invention relates to methods of making compounds of the formula (VI-1) wherein the method comprises reacting a compound of the formula (VII-1)

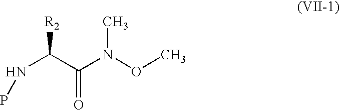

with a Grinard reagent formed in situ by addition of 2-(2-bromo-ethyl)-[1,3]dioxane to a mixture comprising magnesium, alkylmagnesium halide, and the compound of the formula (VII-1).

In another preferred embodiment, the methods further comprise formation of the compound of the formula (VII-1) by coupling N,O-dimethylhydroxylamine hydrochloride and a compound of the formula (VIII-1)

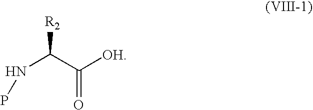

In various preferred embodiments of the above-described methods, the acid catalyst comprises p-toluenesulfonic acid, methanesulfonic acid, or sulfuric acid; the reducing agent comprises aluminum triisopropoxide and isopropanol or N-Selectride; and/or the silylating agent comprises 1,1,1,3,3,3-hexamethyl-disilazane.

Furthermore, in additional aspects, the present invention relates to the compounds herein described including compounds of the formula (VIg-1), (VIf-1), (VIe-1), (VId-1), (VIb-1), (VIa-1), (VI-1), and (VII-1).

In some preferred embodiments of the methods and compounds of aforementioned aspects of the present invention, P is carbobenzyloxy, t-butoxy carbonyl or 9-fluorenyl-methylenoxy carbonyl, preferably, t-butoxy carbonyl.

In some preferred embodiments of the methods and compounds of the aforementioned aspects of the present invention, R₂ is 3-fluoro-benzyl.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

Before the present compounds and methods are disclosed and described, it is to be understood that this invention is not limited to specific synthetic methods of making that may of course vary. It is also to be understood that the terminology used herein is for the purpose of describing particular embodiments only and is not intended to be limiting.

In this specification and in the claims that follow, reference will be made to a number of terms that shall be defined to have the following meanings:

Unless otherwise indicated, the alkyl and alkenyl groups referred to herein, as well as the alkyl moieties of other groups referred to herein (e.g., alkoxy), may be linear or branched, and they may also be cyclic (e.g., cyclopropyl, cyclobutyl, cyclopentyl, cyclohexyl or cycloheptyl) or be linear or branched and contain cyclic moieties. Such alkyl and alkoxy groups may be substituted with one, two or three halogen and/or hydroxy atoms, preferably fluorine atoms.

Unless otherwise indicated, “halogen” and “halide” includes fluorine, chlorine, bromine, and iodine.

“(C₃-C₁₀)cycloalkyl” when used herein refers to cycloalkyl groups containing zero to two levels of unsaturation such as cyclopropyl, cyclobutyl, cyclopentyl, cyclopentenyl, cyclohexyl, cyclohexenyl, 1,3-cyclohexadiene, cycloheptyl, cycloheptenyl, bicyclo[3.2.1]octane, norbornanyl, and the like.

“(C₂-C₉)heterocycloalkyl” when used herein refers to pyrrolidinyl, tetrahydrofuranyl, dihydrofuranyl, tetrahydropyranyl, pyranyl, thiopyranyl, aziridinyl, oxiranyl, methylenedioxyl, chromenyl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, chromanyl, and the like. One of ordinary skill in the art will understand that the connection of said (C₂-C₉)heterocycloalkyl rings is through a carbon or a sp³ hybridized nitrogen heteroatom.

“(C₂-C₉)heteroaryl” when used herein refers to furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5, 6, 7, 8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl, benzoxazinyl, and the like. One of ordinary skill in the art will understand that the connection of said (C₂-C₉)heterocycloalkyl rings is through a carbon atom or a sp³ hybridized nitrogen heteroatom.

“Aryl” when used herein refers to phenyl or naphthyl.

“Protected amine” and “protected amino” refers to an amine group with one of the hydrogen atoms replaced with a protecting group (P). Any suitable protecting group may be used for amine protection. Suitable protecting groups include carbobenzyloxy, t-butoxy carbonyl (BOC) or 9-fluorenyl-methylenoxy carbonyl.

By “pharmaceutically acceptable” is meant a material that is not biologically or otherwise undesirable, i.e., the material may be administered to an individual along with the selected compound without causing any undesirable biological effects or interacting in a deleterious manner with any of the other components of the pharmaceutical composition in which it is contained.

The term “subject” is meant an individual. Preferably, the subject is a mammal such as a primate, and more preferably, a human. Thus, the “subject” can include domesticated animals, livestock, and laboratory animals.

In general, “effective amount” or “effective dose” means the amount needed to achieve the desired result or results (treating or preventing the condition). One of ordinary skill in the art will recognize that the potency and, therefore, an “effective amount” can vary for the various compounds used in the invention. One skilled in the art can readily assess the potency of the compounds.

Unless otherwise noted, numerical values described and claimed herein are approximate. Variation within the values may be attributed to equipment calibration, equipment errors, purity of the materials, among other factors. Additionally, variation may be possible, while still obtaining the same result.

The compounds and processes of the present invention are useful in the manufacture of dihydroxyhexanoic acid derivatives. The present invention includes methods of making a compound of the formula (V-1) as shown in Scheme 1.

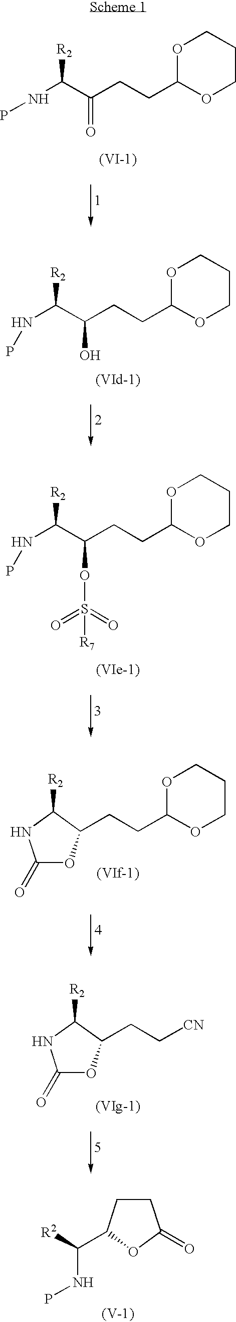

In Scheme 1 step 1, a compound of the formula (VI-1) is reduced with a reducing agent to form a compound of the formula (VId-1). In one embodiment, the reducing agent is aluminum triisopropoxide and isopropanol. Preferably, the temperature is maintained above room temperature, more preferably between about 60° C. and about 82° C. The product alcohol can be isolated by either cooling the reaction mixture to room temperature, diluting with more isopropanol and collecting the crystalline material or by cooling the reaction to room temperature and adding 1N HCL and water and collecting the crystalline material.

Step 2 of Scheme 1 includes reacting a compound of the formula R₇—SO₂-halide and a compound of the formula (VId-1) in the presence of a base to form the compound of the formula (VIe-1). Any amine base is suitable, including pyridine, triethylamine, N-methylmorpholine, and diisopropylethylamine. In one embodiment, R₇—SO₂-halide is p-toluenesulfonyl chloride or methanesulfonyl chloride. In another embodiment, the conversion of hydroxy dioxane (VId-1) to dioxane oxazolidinone (VIe-1) can be achieved by treatment of the hydroxy dioxane (VId-1) with methanesulfonyl chloride and triethylamine in tetrahydrofuran solution and heating the mixture to cause the cyclization of the mesylate formed in situ to the oxazolidinone.

In step 3 of Scheme 1, a compound of the formula (VIf-1) may be formed by heating the compound of the formula (VIe-1). The reaction may proceed by dissolving compound VIe-1 in a solvent such as pyridine or N-methyl imidazole and heating the mixture for several hours at temperature from above room temperature, preferably from about 50° C. to about 100° C.; preferably at about 80° C. The oxazolidinone (VIf-1) may be recovered by extraction into an organic solvent such as ethyl acetate and removal of the amine solvents by extraction of the solution with aqueous acid.

Step 4 of Scheme 1 depicts reacting hydroxylamine hydrochloride, an acid catalyst, and a compound of the formula (VIf-1) to form a compound of the formula (VIg-1). In one embodiment, the acid catalyst is p-toluenesulfonic acid, methanesulfonic acid, or sulfuric acid. The reaction may occur in a solvent, such as ethanol. In one embodiment, the reaction occurs in methanol with tosic acid at reflux for 8 to 24 hours. The resulting nitrile oxazolidinone contains a small amount of the corresponding ethyl ester which is not removed since it also is converted to the desired lactone in subsequent steps.

Step 5 of Scheme 1 includes a) hydrolyzing a compound of the formula (VIg-1) with an aqueous solution in the presence of a base, b) protecting the amine group of the compound so formed, and c) cyclizing the compound so formed with heat and an acid catalyst. In one embodiment, the compound VIg-1 is hydrolyzed with sodium hydroxide. The pH is adjusted to approximately 10 and tetrahydrofuran and BOC dicarbonate are added. This provides the protected hydroxy acid, which may be heated in 10% acetic acid and toluene to provide the protected amine lactone (V-1).

The compound of formula (V-1) may also be produced according to Scheme 2.

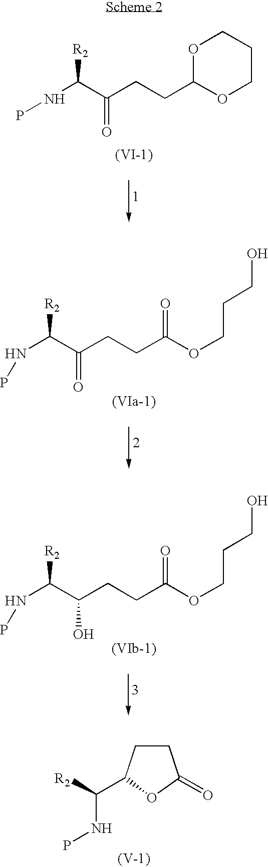

In step 1 of Scheme 2, a compound of the formula (VI-1) may be reacted with ozone to form a compound of the formula (VIa-1). The compound VI-1 may be present in a solvent, such as ethyl acetate, and the ozone introduced through sparging at a temperature below room temperature, preferably at about −15° C., until the starting dioxane ketone is substantially reacted. Any excess ozone may be removed by bubbling nitrogen through the solution. The resulting crude ketone ester mixture may be isolated after treatment with aqueous sodium bisulfite to remove any hydroperoxides.

Alternatively, in step 1 of Scheme 2, the compound of the formula (VIa-1) may be formed by reacting hypochlorous acid and a compound of the formula (VI-1). Such an oxidation reaction typically produces chlorinated forms of the compound Vial as side products in addition to the compound VIa-1. This oxidation reaction proceeds by mixing the compound VI-1 in solvent, such as acetic acid and/or acetone, and adding sodium hypochlorite, while keeping the mixture at a low temperature, preferably at or below about 0° C.

As a means to convert the side product chlorinated forms of the compound VIa-1 to compounds of the formula V-1, the compounds formed from the hypochlorous acid oxidation reaction may optionally be hydrogenated by reaction with hydrogen in the presence of a catalyst. The hydrogenation may include introducing the products from the hypochlorous acid oxidation reaction into a solvent system of tetrahydrofuran and water, followed by addition of a palladium on carbon catalyst. The resulting mixture is subjected to hydrogen above atmospheric pressure and temperature. In one embodiment, the pressure is about 80 pounds per square inch and the temperature is maintained from about 60° C. to about 70° C. until the reaction is substantially complete.

In step 2 of Scheme 2, the compound of the formula (VIb-1) may be formed by reacting a silylating agent and a compound of the formula (VIa-1) and reacting the compound so formed with a reducing agent. In one embodiment, the reducing agent is N-Selectride. In another embodiment, the silylating agent is 1,1,1,3,3,3-hexamethyl-disilazane. The reduction reaction may occur at temperatures below about 0° C., preferably below about −20° C., more preferably below about −50° C. In addition, the reducing agent may be present in slight excess.

In step 3 of Scheme 2, the compound of the formula (V-1) is formed by heating a compound of the formula (VIb-1) in the presence of an acid catalyst, such as acetic acid. In one embodiment, the cyclization reaction occurs by introducing the compound VIb-1 into a solvent mixture, such as toluene and 10% acetic acid, at the solvent reflux temperature for 8 to 16 hours. This provides the desired lactone as a crystalline solid after work up.

One method of making the compound of the formula (VI-1) is by reacting a compound of the formula (VII-1)

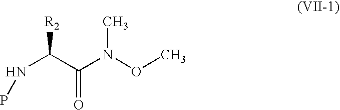

with a Grinard reagent formed in situ by addition of 2-(2-bromo-ethyl)-[1,3]dioxane to a mixture comprising magnesium, alkylmagnesium halide, and the compound of the formula (VII-1). In one embodiment, the alkylmagnesium halide comprises methyl magnesium chloride and/or methyl magnesium bromide in a solvent. Any exotherm formed from the reaction may be controlled by the rate of addition of the bromide.

The compound of the formula (VII-1) may be formed by coupling N,O-dimethylhydroxylamine hydrochloride and a compound of the formula (VIII-1)

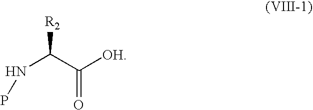

This coupling reaction may be performed by mixed anhydride procedure. In one mixed anhydride procedure, compound VIII-1 is combined with methylene chloride and N-methylmorpholine is added followed by isobutyl chloroformate. In a separate mixture, a slurry of N,O-dimethylhydroxylamine hydrochloride is treated with N-methylmorpholine. The two reaction mixtures are combined and then quenched with a solution of citric acid in water. This procedure preferably operates at a temperature below about 20° C., more preferably below about 0° C.

Unless indicated otherwise, the pressure of each of the above reactions is not critical. Generally, the reactions are conducted at a pressure of about one to about three atmospheres, preferably at ambient pressure (about one atmosphere).

The compounds of the formulas (VIg-1) and (VIf-1) are capable of forming a wide variety of different salts with various inorganic and organic acids.

The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [i.e., 1,1′-methylene-bis-(2-hydroxy-3-naphthoate)] salts.

The compounds of the formulas (VIg-1) and (VIf-1) may be capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those that form non-toxic base salts with the herein described compounds of formula Ia-1. These non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

The compounds of the present invention are important in the manufacture of dihydroxyhexanoic acid derivatives of the formula Ia-1:

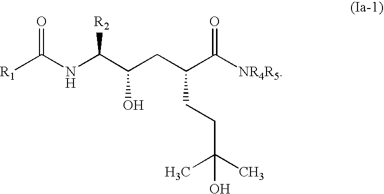

The compound of the formula Ia-1 and its pharmaceutically acceptable salts (hereinafter also referred to, collectively, as “the active compounds”) are potent antagonists of the CCR1 receptors. The active compounds are useful in the treatment or prevention of autoimmune diseases (such as rheumatoid arthritis, type I diabetes (recent onset), inflammatory bowel disease, optic neuritis, psoriasis, multiple sclerosis, polymyalgia rheumatica, uveitis, and vasculitis), acute and chronic inflammatory conditions (such as osteoarthritis, adult respiratory distress syndrome, Respiratory Distress Syndrome of infancy, ischemia reperfusion injury, and glomerulonephritis), allergic conditions (such as asthma and atopic dermatitis), infection associated with inflammation (such as viral inflammation (including influenza and hepatitis) and Guillian-Barre), transplantation tissue rejection, atherosclerosis, restenosis, HIV infectivity (co-receptor usage), and granulomatous diseases (including sarcoidosis, leprosy and tuberculosis).

The activity of the compounds of the formula Ia-1 can be assessed according to procedures known to those of ordinary skill in the art. Examples of recognized methods for determining CCR1 induced migration can be found in Coligan, J. E., Kruisbeek, A. M., Margulies, D. H., Shevach, E. M., Strober, W. editors: Current Protocols In Immunology, 6.12.1-6;12.3. (John Wiley and Sons, NY, 1991). One specific example of how to determine the activity of a compound for inhibiting migration is described in detail below.

Chemotaxis Assay:

The ability of compounds to inhibit the chemotaxis to various chemokines can be evaluated using standard 48 or 96 well Boyden Chambers with a 5 micron polycarbonate filter. All reagents and cells can be prepared in standard RPMI (BioWhitikker Inc.) tissue culture medium supplemented with 1 mg/ml of bovine serum albumin. Briefly, MIP-lx (Peprotech, Inc., P.O. Box 275, Rocky Hill N.J.) or other test agonists, were placed into the lower chambers of the Boyden chamber. A polycarbonate filter was then applied and the upper chamber fastened. The amount of agonist chosen is that determined to give the maximal amount of chemotaxis in this system (e.g., 1 nM for MIP-1 (x should be adequate).

THP-1 cells (ATCC TIB-202), primary human monocytes, or primary lymphocytes, isolated by standard techniques can then be added to the upper chambers in triplicate together with various concentrations of the test compound. Compound dilutions can be prepared using standard serological techniques and are mixed with cells prior to adding to the chamber.

After a suitable incubation period at 37 degrees centigrade (e.g. 3.5 hours for THP-1 cells, 90 minutes for primary monocytes), the chamber is removed, the cells in the upper chamber aspirated, the upper part of the filter wiped and-the number of cells migrating can be determined according to the following method.

For THP-1 cells, the chamber (a 96 well variety manufactured by Neuroprobe) can be centrifuged to push cells off the lower chamber and the number of cells can be quantitated against a standard curve by a color change of the dye fluorocein diacetate.

For primary human monocytes, or lymphocytes, the filter can be stained with Dif Quik® dye (American Scientific Products) and the number of cells migrating can be determined microscopically.

The number of cells migrating in the presence of the compound are divided by the number of cells migrating in control wells (without the compound). The quotient is the % inhibition for the compound which can then be plotted using standard graphics techniques against the concentration of compound used. The 50% inhibition point is then determined using a line fit analysis for all concentrations tested. The line fit for all data points must have a coefficient of correlation (R squared) of >90% to be considered a valid assay.

The compounds of formula Ia-1 had IC₅₀ values of less than 25 μM, in the Chemotaxis assay.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation. The active compounds of the invention may also be formulated for sustained delivery. Formulations of dihydroxyhexanoic acid derivatives are exemplified in co-pending U.S. patent application serial Nos. 60/300,255; 60/300,261; 60/300,256; and 60/300,260, all of which were filed on Jun. 22, 2001 and all of which are incorporated herein in their entireties for all purposes.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glyqolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection; may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., rheumatoid arthritis) is 0.1 to 1000 mg of the active ingredient per unit dose which could be administered, for example, 1 to 4 times per day.

Aerosol formulations for treatment of the conditions referred to above (e.g., rheumatoid arthritis) in the average adult human are preferably arranged so that each metered dose or “puff” of aerosol contains 20 μg to 1000 μg of the compound of the invention. The overall daily dose with an aerosol will be within the range 0.1 mg to 1000 mg. Administration may be several times daily, for example 2, 3, 4 or 8 times, giving for example, 1, 2 or 3 doses each time.

The active agents can be formulated for sustained delivery according to methods well known to those of ordinary skill in the art. Examples of such formulations can be found in U.S. Pat. Nos. 3,538,214, 4,060,598, 4,173,626, 3,119,742, and 3,492,397.

The compounds of the invention can also be utilized in combination therapy with other therapeutic agents such as with immunosuppressant agents such as cyclosporin A and FK-506, Cellcept®, rapamycin, leuflonamide or with classical antiinflammatory agents (e.g. cyclooxygenase/lipoxegenase inhibitors) such as tenidap, aspirin; acetaminophen, naproxen and piroxicam, steroids including prednisone, azathioprine and biological agents such as OKT-3, anti IL-2 monoclonal antibodies (such as TAC).

Experimental

The following examples are put forth so as to provide those of ordinary skill in the art with a complete disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were utilized without further purification. The following abbreviations are herein used:

AA is amino acid

AcOH is acetic acid

Boc is t-butoxy carbonyl

CDCl₃ is deuteriotrichloromethane

DMF is N,N-dimethylformamide

EtOAc is ethyl acetate

HCl is hydrochloric acid

HMDS is hexamethyldisilazane

IPE is isopropyl ether

MeOH is methanol

THF is tetrahydrofuran

g is grams

L is liter

M is molar

ml is milliliter

mmol is millimole

MHz is mega hertz

N is normal

psi is pounds per square inch

h is hours

min is minutes

sec is seconds

mp is melting point

RT is room temperature

Vacuo is in vacuum

˜ is roughly approximate to*

HPLC is high pressure liquid chromatography

LCMS is liquid chromatograph mass spectrometer

NMR is nuclear magnetic resonance

TLC is thin layer chromatography * Note that all numbers provided herein are approximate, but effort have been made to ensure accuracy with respect to numbers (e.g., amounts, temperature, etc.); however some errors and deviations should be accounted for.

### EXAMPLE 1

### N′-Methoxy-N-methyl (S)-N-BOC-(3-fluorophenyl)alanine Amide (VII-3)

In a 1L 4N-flask equipped with addition funnel, cold temperature thermometer, under nitrogen gas and mechanical stirrer, combined BOC-(3-fluorophenyl)alanine (VIII-3) (56.6 g, 0.2 moles) and methylene chloride. This was stirred to give a solution and N-methylmorpholine (23.05 ml, 0.21 moles) was added with a slight exotherm of 18° C. to 24° C. The solution was cooled to −25° C. and isobutyl chloroformate (25.27 ml, 0.195 moles) was added over a 2 to 3 minute period while keeping the temperature from −25° C. to −20° C. A precipitate formed as the reaction mixture was stirred at −20° C. to −10° C. for one hour. In a separate flask, a slurry of N,O-dimethylhydroxyiamine hydrochloride (21.45 g, 0.22 moles) was treated with N-methylmorpholine (24.15 ml, 0.22 moles) at room temperature. The reaction remained a slurry throughout as the N-methylmorpholine hydrochloride formed. After one hour, the hydroxylamine suspension was added over 30 minutes to the mixed anhydride with the temperature rising to 5° C. The mixture was stirred at room temperature over the weekend.

The reaction was quenched with a solution of citric acid (50 g) in water (200 ml). The organic layer was separated and washed with water, saturated sodium bicarbonate solution and brine. The organics were dried over magnesium sulfate filtered and evaporated to an oil, which was pumped at high vacuum to remove residual solvents. The crude yield was 61.7 g, 97% by weight and 90-92% corrected for HPLC purity assay. The NMR showed the presence of an impurity which was thought to be the N,O-dimethylhydroxylamine sec-butyl carbamate. This was suitable for use in the next step without purification. HPLC: 97.33% desired amide; 1.57% enantiomer. (Starting BOC-AA was 1.7% enantiomer. [a]D: 21.8 (c=1.0, methylene chloride).

¹H NMR (CDCl₃, 400 MHz): δ 7.22 (q, 1), 6.93-6.85 (m, 3), 5.24 (m, 1), 4.91 (m, 1), 3.66 (s, 3), 3.15 (s, 3), 3.02 (abx, 1), 2.84 (abx, 1), 1.36 (s, 9). ¹³C NMR (CDCl₃, 100 MHz): 172.1, 164.1, 155.3, 129.985, 129.903, 125.345, 125.323, 116.663, 116.454, 113.963. 113.754, 79.9, 61.8, 51.6, 38.7, 32.3, 28.5.

LCMS exact mass measurement: C₁₆H₂₃N₂O₄F+Na+; Calcd: 349.1540; Measured: 349.1548.

### EXAMPLE 2

### [4-[1,3]Dioxan-2-yl-(1S)-(3-fluoro-benzyl)-2-oxo-butyl]-carbamic Acid Tert-Butyl Ester (VI-3)

N′-Methoxy-N′-methyl (S)-N-BOC-(3-fluorophenyl)alanine amide (VII-3) (61.4 g, 0.188 moles) was dissolved in tetrahydrofuran (300 ml) in a 1L flask under nitrogen. Magnesium turnings (6.76 g, 0.282 moles) were added and the mixture was cooled to −10° C. Methyl magnesium chloride (62.6 ml, 3M in THF, 0.188 moles) was added dropwise over 25 minutes with the temperature below −5° C. The reaction was then stirred for about 10 minutes until the gas evolution stopped (methane) and the temperature was 0° C. The reaction was allowed to warm to 13° C. and 2-(2-bromoethyl)-1,3-dioxane (25 ml, 0.18 moles) was added over a two minute period. The Grignard reaction initiated quickly and the mild exotherm was controlled by external cooling to less than 28° C. [On larger scale, the exotherm can be partially controlled by the addition of the bromide.] The reaction was then stirred over night at room temperature.

After an aliquot was quenched, NMR analysis showed significant starting Weinreb amide. Magnesium turnings (5 g, 0.2 moles) were added followed by 2-(2-bromoethyl)-1,3-dioxane (13.84 ml, 0.1 moles) and the temperature again controlled to less than 28° C. After an additional hour, an aliquot showed a small amount of Weinreb amide and a final charge of bromide (6.5 ml, 0.05 moles) was added. The total amount of bromide was 0.2185 moles or 1.75 equivalents. The reaction was stirred overnight to ensure completion. As much as 2 equivalents of bromide and magnesium have been required to fully consume the Weinreb amide starting material.

The reaction was quenched with a solution of ammonium chloride (165 g) in water (1L) and ethyl acetate (400 ml) was added. The layers were stirred for 10 minutes and separated. The aqueous was extracted with ethyl acetate (400 ml). The combined organics were washed with water (2 times 400 ml) and brine. The solution was dried with magnesium sulfate, filtered and evaporated to a waxy, yellow solid. The solid was stirred with IPE (150 ml) for 30 minutes, then hexanes (200 ml) were added and the slurry was stirred for two hours. The solids were collected and washed with hexanes (2 times 100 ml). After drying, the solids weighed 49.66 g, 76% yield, based on starting Weinreb amide or 65% based on starting BOC-amino acid. mp 107-108-5° C. [α]_{D}: −36.8 (c=1.0, MeOH).

HPLC: 99.817% product; 0.0293% enantiomer; 0.15% Weinreb amide.

¹H NMR (CDCl₃, 400 MHz): δ 7.21 (m, 1), 6.85 (m, 3), 5.18 (d, 1), 4.52 (m, 2), 4.01 (m, 2), 3.68 (m, 2), 3.10 (m, 1), 2.87 (m, 1), 2.56 (m, 2), 2.00 (m, 1), 1.85 (m, 2), 1.37 (m, 10). ¹³C NMR (CDCl₃, 100 MHz): 164.2, 155.3, 139.187, 139.112, 130.206, 130.124, 125.233, 125.207, 116.614, 116.405, 114.105, 113.896, 100.7, 80.0, 67.0, 60.2, 37.4, 34.4, 29.0, 28.5, 25.9.

Anal. Calcd. for C₂₀H₂₈NO₅F: C, 62.98; H, 7.398; N, 3.67. Found: C, 62.77; H, 7.52; N, 3.67.

### EXAMPLE 3

### [4-[1,3]Dioxan-2-yl-(1S)-(3-fluoro-benzyl)-(2R)-hydroxy-butyl]-carbamic Acid Tert-Butyl Ester (VId-3)

[4-[1,3]Dioxan-2-yl-(1S)-(3-fluoro-benzyl)-2-oxo-butyl]-carbamic acid tert-butyl ester (VI-3) (7.5 g, 19.7 mmol) and aluminum triisopropoxide (4.4 g, 21.6 mmol) were added to isopropanol (60 ml) and the mixture was heated to reflux for 0.5 h. The reaction was cooled to room temperature with an ice water bath. At this point, two, different work ups were possible.

A) The reaction was diluted first with 1N HCl (120 ml) and then water was added to ca. 400 ml volume. After stirring for one hour, the solids were collected, washed with water and dried. This provided the desired alcohol, 6.63 g, 88% yield. The material was slightly colored, but suitable for the next reaction.

B) The cooled reaction mixture was diluted with isopropanol (60 ml) and stirred for one hour. The solids were collected and washed with isopropanol (3 times 15 ml) and hexanes and air-dried. The yield was 5.8 g, 77% of white solids with only trace impurities. mp 180.5-181.5° C. [α]_{D}: −16.1 (c=1.0, CH₂Cl₂).

¹H NMR (CDCl₃, 400 MHz): δ 7.22 (m, 2), 6.98 (d, 1), 6.94-6.86 (m, 2), 4.60 (m, 2), 4.15 (m, 2), 3.78 (m, 3), 3.61 (s, 1), 3.46 (s, 1), 2.91 (m, 1), 2.75 (m, 1), 2.16 (m, 1), 1.84-1.55 (m, 4), 1.34 (s, 9). ¹³C NMR (CDCl₃, 100 MHz): 164.2, 161.8, 156.0, 141.4, 129.944, 129.862, 125.3, 116.659, 116.45, 113.399, 113.189, 112.5, 102.1, 79.6, 73.7, 67.2, 56.3, 35.6, 32.1, 28.5, 27.9, 25.8.

Anal. Calcd. for C₂₀H₃₀NO₅F: C, 62.643; H, 7.885; N, 3.65. Found: C, 62.60; H, 8.00; N, 3.64.

### EXAMPLE 4

### Methanesulfonic Acid (2S)-tert-butoxycarbonylamino-1-(2-[1,3]dioxan-2-ylethyl)-(3R)-(3-fluoro-phenyl)-propyl Ester (VIe-3)

[4-[1,3]Dioxan-2-yl-(1S)-(3-fluoro-benzyl)-(2R)-hydroxy-butyl]-carbamic acid tert-butyl ester (VId-3) (30 g, 78.3 mmol) was added to pyridine (100 ml) under nitrogen. Methanesulfonyl chloride (6.67 ml, 86 mmol) was added dropwise over 35 min with a mild exotherm from 20° C. to 27° C. The reaction mixture was stirred for 2.5 h at which time a thin-layer chromatography showed the conversion was complete. The reaction was cooled to 15° C. in an ice water bath and water (250 ml) was added slowly with a slight initial exotherm. The resulting slurry was stirred at 15° C. for 20 min and the solids were collected and washed with water. The solids were air-dried and then slurried in isopropyl ether (275 ml) for 5 h. The product was filtered and washed with IPE and dried. The yield was 34 g, 94%. mp 121-22° C. [α]_{D}: −4.2 (c=1.0, MeOH).

¹H NMR (CDCl₃, 400 MHz): δ 7.24 (m, 1), 6.98 (d, 1), 6.91 (m, 2), 4.86 (s, 1)ₘ, 4.80 (d, 1), 4.10 (m, 3), 3.75 (t, 1), 3.07 (s, 3), 2.95 (dd, 1), 2.64 (m, 1), 2.05 (m, 1), 1.871.68 (m, 4), 1.32 (s, 9). ¹³C NMR (CDCl₃, 100 MHz): 164.2, 161.8, 155.5, 140.152, 140.077, 130.187, 130.105, 125.0, 116.379, 116.166, 113.818, 113.608, 101.1, 84.4, 79.9, 67.1, 54.0, 38.9, 35.5, 31.0, 28.4, 25.9, 25.5.

Anal. Calcd. for C₂₁H₃₂NO₇FS: C, 54.647; H, 6.987; N, 3.035. Found: C, 54.32; H, 7.08; N, 2.96.

### EXAMPLE 5

### 5S-(2-[1,3]Dioxan-2-yl-ethyl)-(4S)-(3-fluoro-benzyl)-oxazolidin-2-one (VIf-2)

Methanesulfonic acid (2S)-tert-butoxycarbonylamino-1-(2-[1,3]dioxan-2-ylethyl)-(3R)-(3-fluoro-phenyl)-propyl ester (VIe-3) (29 g, 62.0 mmol) was dissolved in dry pyridine (50 ml) under nitrogen and the mixture was heated in an oil bath to 80° C. After 2 h, the reaction solution was cooled to RT and ethyl acetate and water were added. The layers were separated and the aqueous layer was extracted with ethyl acetate. The combined organics were washed with 3N HCl (2 times 300 ml), water, and brine. The solution was dried over sodium sulfate. The solution was filtered and evaporated in vacuum to provide the desired product as an oil in quantitative yield, 20.1 g. This was suitable for use in the next step. [α]_{D}: −47.7 (c=1.0, MeOH).

¹H NMR (CDCl₃, 400 MHz): δ 7.24 (m, 1), 6.91 (m, 2), 6.84 (d, 1), 6.60 (s, 1), 4.45 (s, 1), 4.26 (s, 1), 3.98 (m, 2), 3.67 (m, 3), 2.78 (m, 2), 1.95 (m, 1), 1.71-1.53 (m, 4), 1.28 (d, 1). ¹³C NMR (CDCl₃, 100 MHz): 164.3, 161.9, 159.4, 138.742, 138.667, 130.655, 130.569, 135.184, 135.158, 116.476, 116.267, 114.375, 114.165, 101.3, 81.4, 67.0, 59.0, 41.2, 30.3, 29.0, 25.9.

LCMS exact mass measurement: C₁₆H₂₀NO₄F+Na+; Calcd: 332.1274; Measured 332.1262.

### EXAMPLE 6

### 3-[(4S)-(3-Fluoro-benzyl)-2-oxo-oxazolidin-(5S)-yl]-propionitrile (VIg-2)

5S-(2-[1,3]Dioxan-2-yl-ethyl)-(4S)-(3-fluoro-benzyl)-oxazolidin-2-one (VIf-2) (19.2 g, 62.13 mmol) was combined with hydroxylamine hydrochloride (5.61 g, 80.77 mmol), p-toluenesulfonic acid (“tosic acid”) (1.18 g, 6.43 mmol) in ethanol (200 ml) and heated to reflux overnight. The reaction was concentrated in vacuo and the resulting oil was partitioned between ethyl acetate and water. The layers were separated and the organics were washed with water and brine and concentrated to an oil in vacuo (vacuum). The oil was stirred with IPE (80 ml) for 2 h during which time a solid formed. The suspension was diluted with an equal volume of hexanes. The solids were collected by filtration and washed with hexanes and were dried to yield the desired nitrile, 13.0 g, 90%. mp 68.5-70° C. [α]_{D}: −67.3 (c=1.0, MeOH).

¹H NMR (CDCl₃, 400 MHz): δ 7.30 (m, 1), 6.97 (m, 2), 6.91 (d, 1), 6.41 (s, 1), 4.36 (m, 1), 3.71 (m, 1), 2.95-2.82 (m, 2), 2.46 (t, 2), 1.93 (m, 1), 1.76 (m, 1). ¹³C NMR (CDCl₃, 100 MHz): 164.5, 162.0, 137.994, 137.92, 131.029, 130.946, 125.106, 125.072, 116.401, 116.192, 114.861, 114.651, 79.5, 58.8, 41.1, 30.7, 13.6.

LCMS exact mass measurement: C₁₃H₁₃N₂O₂F+Na+; Calcd: 249.1039; Measured: 249.1027.

### EXAMPLE 7

### [(2S)-(3-Fluoro-phenyl)-(1S)-(5-oxo-tetrahydro-furan-2-yl)-ethyl]-carbamic Acid Tert-Butyl Ester (V-3)

3-[(4S)-(3-Fluoro-benzyl)-2-oxo-oxazolidin-(5S)-yl]-propionitrile (VIg-2) (10.7 g, 43.15 mmol) was dissolved in 50% aqueous ethanol (100 ml) with sodium hydroxide (6.9 g, 172 mmol) and was heated to reflux overnight. The reaction was cooled to room temperature. The solution was concentrated in vacuo, more water was added and the solution was concentrated again to remove the ethanol. The final oil was diluted to 75 ml with water. The aqueous solution was cooled in an ice bath and concentrated HCl was added to adjust the pH to 10.0. Tetrahydrofuran (50 ml) was added to the reaction and t-butoxy carbonyl dicarbonate (12.22 g, 56 mmol) was added. The pH was monitored and additional sodium hydroxide pellets were added to maintain pH greater than 9.0. After stirring overnight, the THF was evaporated in vacuo and the aqueous solution was extracted with ethyl acetate twice. This was found to contain excess BOC dicarbonate. The aqueous layer was acidified with acetic acid (10 ml) and extracted twice with ethyl acetate. The combined ethyl acetate layers were evaporated and the solid residue was dissolved in toluene (125 ml) and acetic acid (5 ml) and heated to reflux for 3 h. This was cooled, diluted with ethyl acetate (125 ml) and water and sodium bicarbonate was added to neutralize the acetic acid. The organic layer was evaporated in vacuo to provide the desired lactone as a solid; 12.58 g, 90.3% yield. mp 108-108.5° C. [α]_{D}: −26.1 (c=1.0, MeOH). HPLC: 99.8% desired diastereomer.

¹H NMR (CDCl₃, 400 MHz): δ 7.22 (m, 1), 6.98 (d, 1), 6.90 (m, 2), 4.82 (d, 1), 4.46 (t, 1), 3.97 (q, 1), 2.88 (m, 2), 2.49 (m, 2), 2.11 (m, 2), 1.34 (s, 9). ¹³C NMR (CDCl₃, 100 MHz): 177.4, 164.3, 161.9, 156.1, 140.096, 140.025, 130.344, 130.262, 125.229, 125.203, 116.513, 116.304, 113.963, 113.754, 80.4, 80.2, 54.1, 39.3, 28.9, 28.4, 24.3.

Anal. Calcd. for C₁₇H₂₂NO₄F: C, 63.14; H, 6.86; N, 4.33; F, 5.88. Found: C, 62.98; H, 6.85; N, 4.14; F, 5.95.

### EXAMPLE 8

### (5S)-tert-Butoxycarbonylamino-6-(3-fluoro-phenyl)₄-oxo-hexanoic Acid 3-hydroxy-propyl Ester (VIa-3)

[4-[1,3]Dioxan-2-yl-(1S)-(3-fluoro-benzyl)-2-oxo-butyl]-carbamic acid tert-butyl ester (VI-3) (10 g, 0.026 moles) was dissolved in ethyl acetate (100 ml) and cooled to −15° C. Ozone was bubbled through the solution for 55 minutes at which time the starting material was gone by TLC. [Model G1-1 PCI Ozone Generator; run at 95% ozone (where 100% equals a 3% ozone stream in oxygen), 18 psi with air flow at 6 standard cubic feet per hour] The solution was purged with oxygen for several minutes, then water (50 ml) was added followed by sodium bisulfate (2.5 g) and the biphasic mixture was stirred for 30 minutes at which time it was negative to starch iodide paper. The organics were separated, washed with water (2 times) and brine and dried over sodium sulfate for 3 h. The solution was filtered and evaporated to a yellow oil. This was evaporated with methylene chloride several times and used as is for the next reaction.

### EXAMPLE 9

### [(2S)-(3-Fluoro-phenyl)-(1S)-(5-oxo-tetrahydro-furan-2-yl)-ethyl]-carbamic Acid Tert-Butyl Ester (V-3)

(5S)-tert-Butoxycarbonylamino-6-(3-fluoro-phenyl)-4-oxo-hexanoic acid 3-hydroxy-propyl ester (VIa-3) (10.1 g, 0.026 moles) was dissolved in tetrahydrofuran (100 ml) and treated with hexamethyldisilazane (5.8 ml, 0.027 moles) dropwise over 4 minutes. The reaction was heated to a gentle reflux while the nitrogen outflow was monitored for ammonia. After 2.5 h, the ammonia was negligible and the reflux was increased for 30 minutes to remove excess reagent. The reaction was cooled to −78° C. and N-Selectride (31.5 ml, 0.0315 moles) was added dropwise over 35 minutes. After one hour at −78° C., acetic acid (10 ml) in tetrahydrofuran (30 ml) was added dropwise. The reaction was allowed to warm to −10° C. and ethyl acetate (300 ml) and water (300 ml) was added. This was stirred for 10 minutes and the layers were separated. The ethyl acetate layer was washed twice with water. The organics were stirred with aqueous sodium perborate (3 equivalents relative to the Selectride) and the mixture stirred for several hours. The layers were separated and washed with water, with brine, dried over magnesium sulfate and concentrated to an oil in vacuo. The crude oil was dissolved in toluene (100 ml) with acetic acid (10 ml) and the solution was refluxed overnight.

The toluene solution was cooled to room temperature and ethyl acetate (100 ml), water (100 ml) and 6N HCl (15 ml) was added and the mixture was stirred for 30 min. The organics were separated and washed with water (2 times), saturated sodium bicarbonate (2 times), water, and brine. The solution was dried over magnesium sulfate and evaporated to an oil. The oil was dissolved in a 1:1 mixture of ethyl acetate and hexanes and passed through a silica gel pad with the same mixture. The product containing fractions were combined (3-6 of 75-100 ml fractions) and evaporated to an oil which started to crystallize. IPE (30 ml) was added and the slurry stirred for 10 min and hexanes (30 ml) was added. After stirring for an additional 10 min, the solids were collected and washed with hexanes to provide the lactone as a white solid; 5 g, 59% yield. This material was analytically pure by assay and NMR. It was identical with that described above.

### EXAMPLE 10

### [(2S)-(3-Fluoro-phenyl)-(1S)-(5-oxo-tetrahydro-furan-2-yl)-ethyl]-carbamic Acid Tert-Butyl Ester (V-3)

[4-[1,3]Dioxan-2-yl-(1S)-(3-fluoro-benzyl)-2-oxo-butyl]-carbamic acid tert-butyl ester (VI-3) (50 g, 0.13 mol) was dissolved in a mixture of acetic acid (75 mL, 1.31 mol) and acetone (480 mL). The resulting mixture was ccooled to −10° C., and 10% sodium hypochlorite (480 mL, 0.65 mol) was added dropwise while keeping internal reaction temperature under −5° C. After addition, the reaction mixture was allowed to stir at −5° C. to 0° C. for 20 h, HPLC assay of an aliquot showed reaction completion. The reaction was cooled to −10° C., and sodium bisulfite (114 g) solution in 450 mL water was added dropwise while maintaining the reaction temperature below 0° C. The layers were separated, and the aqueous layer was extracted with 2 times 500 mL EtOAc. The organic layers were combined and washed with water (3 times 300 mL). The organic layer was then transferred back to a 3-neck RB flask, to this was added slowly saturated sodium carbonate solution (300 mL), and the resulting mixture was stirred for 30 min to ensure no AcOH was present. (Check pH to be about 7.5, if not, adjust with 3 N HCl). The layers were separated, the organic layer, containing crude (5S)-tert-butoxycarbonylamino-6-(3-fluoro-phenyl)-4-oxo-hexanoic acid 3-hydroxy-propyl ester (VIa-3), was washed with brine solution (500 mL), and concentrated in vacuo as an oil (wt: 53.4 g)

The crude oil that included the compound (VIa-3) obtained from previous step was dissolved in THF (1000 mL), and water (200 mL) was added, followed by 10% Pd/C (5 g). The resulting mixture was subjected to 80 PSI hydrogen at 60-70° C. for 5 h. HPLC assay showed reaction completion, the mixture was filtered, the layers of the filtrate were separated, and the aqueous layer was extracted with EtOAc (2 times 100 mL). The combined organic layers were concentrated under reduced pressure. Toluene (200 mL) was added, and the resulting was concentrated under atmospheric pressure to ˜100 mL, then concentrated in vacuo to give the crude compound (VIa-3) as an oil.

The compound (VIa-3) obtained from the previous step was dissolved in anhydrous THF (500 mL), HMDS (30.5 mL, 0.144 mol) was added. The reaction mixture was heated to a gentle reflux while the nitrogen outflow was monitored for ammonia. After 2.5 h, the reflux was increased for 30 min to remove excess reagent. The reaction was then cooled to −78° C. and N-Selectride (157.2 mL, 0.157 mol) was added dropwise over a period of 40 min (exothermic) while keeping the internal temperature below −70° C. After 1 h, HPLC assay of an aliquot showed reaction completion, acetic acid (50 mL) in THF (150 mL) was added dropwise, the reaction was allowed to warmed to −10° C., and poured into a flask with 1.5 L EtOAc and 1.5 L water with 110 g of sodium perborate tetrahydrate. The mixture was stirred overnight, layers were separated, the organic layer was washed with water (2 times 400 mL) and concentrated under atmospheric pressure to an oil. The crude oil was dissolved in toluene (500 mL) with acetic acid (50 mL). The resulting solution was heated at reflux for 1 h, HPLC assay showed reaction completion. The reaction mixture was concentrated in vacuo to 100 mL, and hexane (500 mL) was added. The mixture was concentrated in vacuo, the resulting solid was triturated with IPE (150 mL) for 1 h, hexane (150 mL) was added, and the mixture stirred for 30 min. The solids were collected by filtration, and rinsed with hexane (150 mL) to afford the lactone as a white solid. (18.2 g). The filtrate was concentrated in vacuo to an oil, then dissolved in 10 mL EtOAc and 40 mL hexane mixture. This was passed through a silica gel (50 g) pad eluting with 20% EtOAc/hexanes (400 mL). (Checked eluate with HPLC assay). The eluate fractions were combined, and concentrated in vacuo to give an oil, 200 mg of the pure lactone collected earlier was added, and the oil started to solidify. 100 mL of IPE was added to triturate for 30 min. This was followed by addition of 100 mL of hexanes, and stirring the mixture for 30 min. The solids were collected, and rinsed with 100 mL hexanes to give additional 13.4 g of the lactone. Combined yield: 31.4 g, 74.2% overall

NMR: Consistent

HPLC: 97% pure, 2.7% diastereomer present, 0.22% enantiomer detected.

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of making a compound of the formula (V-1): 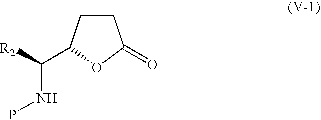
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl(CH₂)ₘ—, (C₃-C₁₀)cycloalkyl-(CH₂)ₘ— or (C₂-C₉)heteroaryl-(CH₂)ₘ— groups may be optionally substituted with one, two, or three substituents independently selected from the group consisting of hydrogen, halogen, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂—N(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂(C₁-C₆)alkyl, (C₁-C₆)alkylHN—SO₂—(C₁-C₆)alkyl, [(C₁-C₆)alkyl]₂N—SO₂—(C₁-C₆)alkyl, QF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4;
wherein the method comprises:
a) hydrolyzing a compound of the formula (VIg-1) 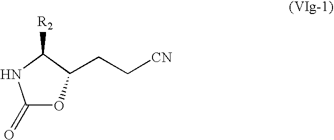
with an aqueous solution in the presence of a base,
d) protecting the amine group of the compound so formed, and
e) cyclizing the compound so formed with heat and an acid catalyst.

2. The method of claim 1, further comprising formation of the compound of the formula (VIg-1) by reacting a compound of the formula (VIf-1) 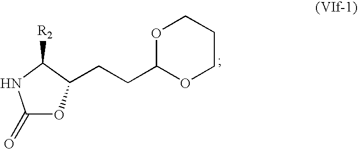
with hydroxylamine hydrochloride and an acid catalyst.

3. The method of claim 2, further comprising formation of the compound of the formula (VIf-1) by heating a compound of the formula (VIe-1) 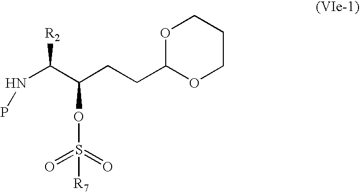
wherein R₇ is (C₁-C₆)alkyl or phenyl wherein the phenyl group may be optionally substituted with one, two, or three (C₁-C₆)alkyl, hydroxy, or halogen groups.

4. The method of claim 3, further comprising formation of the compound of the formula (VIe-1) by reacting a compound of the formhula (VId-1) 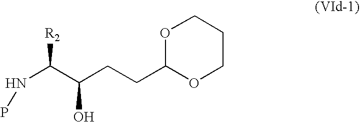
with a compound of the formula R₇—SO₂-halide in the presence of a base.

5. The method of claim 4, further comprising formation of the compound of the formula (VId-1) by reducing a compound of the formula (VI-1) 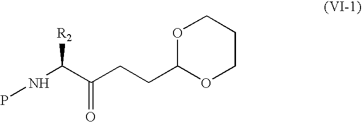
with a reducing agent.

6. A method of making a compound of the formula (V-1): 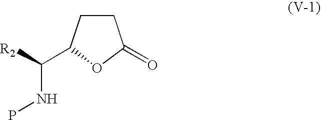
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl-(CH₂)ₘ—, (C₃-C₁₀)cycloalkyl-(CH₂)ₘ— or (C₂-C₉)heteroaryl-(CH₂)ₘ— groups may optionally be substituted with one, two, or three substituents independently selected from the group consisting of hydrogen, halogen, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂NSO₂—, H₂N—SO₂—(C₁-C₆)alkyl, (C₁-C₆)alkylHN—SO₂—(C₁-C₆)alkyl, [(C₁-C₆)alkyl]₂N—SO₂(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4;
wherein the method comprises:
a) reducing a compound of the formula (VI-1) 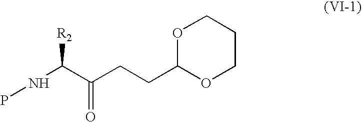
with a reducing agent to form a compound of the formula (VId-1) 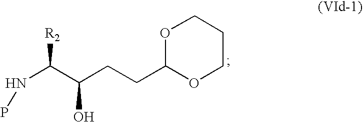
b) reacting the compound of the formula (VId-1) so formed with a compound of the formula R₇—SO₂-halide in the presence of a base to form a compound of the formula (VIe-1) 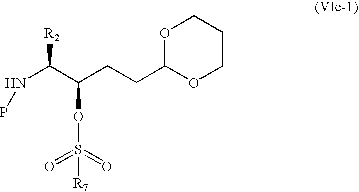
wherein:
R₇ is (C₁-C₆)alkyl or phenyl wherein the phenyl group may be optionally substituted with one, two, or three (C₁-C₆)alkyl, hydroxy, or halogen groups;
c) heating the compound of the formula (VIe-1) so formed to form a compound of the formula (VIf-1) 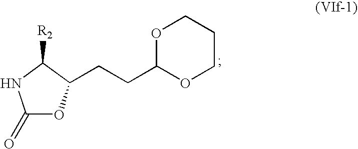
d) reacting the compound of the formula (VIf-1) so formed with an acid catalyst and hydroxylamine hydrochloride to form a compound of the formula (VIg-1) 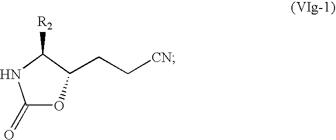
e) hydrolyzing the compound of the formula (VIg-1) so formed with an aqueous solution in the presence of a base;
f) protecting the amine group of the compound so formed; and
g) cyclizing the compound so formed with heat and an acid catalyst.

7. A method of making a compound of the formula (V-1): 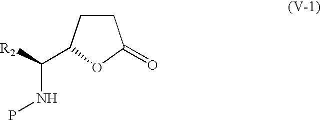
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl(CH₂)ₘ—, (C₃-C₁₀)cycloalkyl-(CH₂)ₘ— or (C₂-C₉)heteroaryl-(CH₂)ₘ— groups may optionally be substituted with one, two, or three substituents independently selected from the group consisting of hydrogen, halogen, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N(C═O)—, H₂N(C═O)—(C₁-C₆)alkyl, (C₁-C₆)alkyl-HN(C═O)—(C₁-C₆)alkyl, [(C₁-C₆)alkyl]₂N(C═O)—(C₁-C₆)alkyl, H(O═C)—NH—, (C₁-C₆)alkyl(C═O)—NH, (C₁-C₆)alkyl(C═O)[N H](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4;
wherein the method comprises heating a compound of the formula (VIb-1) 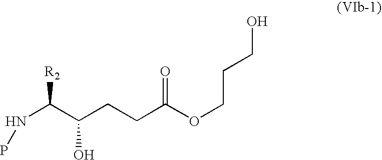
in the presence of an acid catalyst.

8. The method of claim 7, further comprising formation of the compound of the formula (VIb-1) by reacting a compound of the formula (VIa-1) 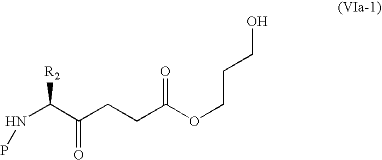
with a silylating agent and further reacting the compound so formed with a reducing agent.

9. The method of claim 8, further comprising formation of the compound of the formula (Via-1) by reacting a compound of the formula (VI-1) 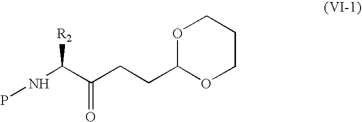
with ozone.

10. The method of claim 8, further comprising formation of the compound of the formula (VIa-1) by reacting a compound of the formula (VI-1) 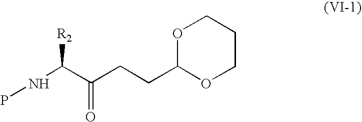
with hypochlorous acid.

11. The method of claim 8, further comprising formation of the compound of the formula (VIa-1) by reacting a compound of the formula (VI-1) 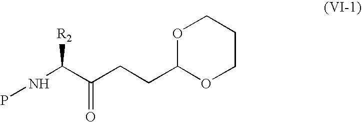
with hypochlorous acid and further reacting the compound so formed with hydrogen in the presence of a catalyst.

12. A method of making a compound of the formula (V-1): 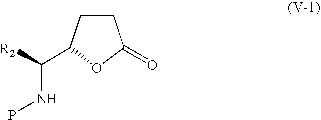
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl(CH₂)ₘ—, (C₃-C₁₀)cycloalkyl-(CH₂)ₘ— or (C₂-C₉)heteroaryl-(CH₂)ₘ-groups may optionally be substituted with one, two, or three substituents independently selected from the group consisting of hydrogen, halogen, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-O(C═O)—, HO—(C═O)—(C₁-C₆)alkyl, (C₁-C₆)alkyl-O—(C═O)—(C₁-C₆)alkyl, (C₁-C₆)alkyl(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—N H—, (C₁-C₆)alkyl(C═O)—N H, (C₁-C₆)alkyl(C═O)-[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂NSO₂—, H₂N—SO₂—(C₁-C₆)alkyl, (C₁-C₆)alkylHN—SO₂—(C₁-C₆)alkyl, [(C₁-C₆)alkyl]₂N—SO₂(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4;
wherein the method comprises:
a) reacting a compound of the formula (VI-1) 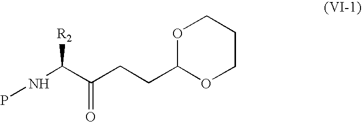
with ozone or hypochlorous acid to form a compound of the formula (VIa-1) 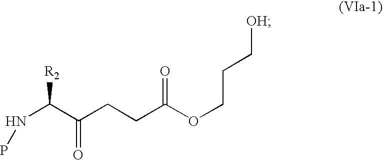
b) reacting the compound of the formula (Via-1) so formed with a silylating agent and further reacting the compound so formed with a reducing agent to form a compound of the formula (VIb-1) 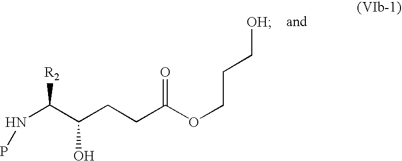
c) heating the compound (VIb-1) so formed in the presence of an acid catalyst.

13. The method of claim 12, further comprising reacting the compound formed in step a) with hydrogen in the presence of a catalyst.

14. A method of making a compound of the formula (VI-1): 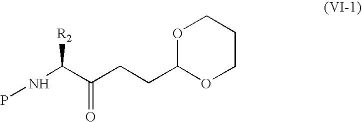
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl20 (CH₂)ₘ—, (C₃-C₁₀)cycloalkyl-(CH₂)ₘ— or (C₂-C₉)heteroaryl-(CH₂)ₘ— groups may optionally be substituted with one, two, or three substituents independently selected from the group consisting of hydrogen, halogen, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-O(C═O)—, HO—(C═O)—(C₁-C₆)alkyl, (C₁-C₆)alkyl-O—(C═O)—(C₁-C₆)alkyl, (C₁-C₆)alkyl(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-N H—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂NSO₂—, H₂N—SO₂—(C₁-C₆)alkyl, (C₁-C₆)alkylH N—SO₂—(C₁-C₆)alkyl, [(C₁-C₆)alkyl]₂N—SO₂(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4;
wherein the method comprises reacting a compound of the formula (VII-1) 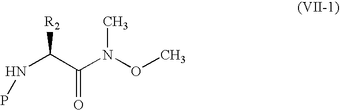
with a Grinard reagent formed in situ by addition of 2-(2-bromo-ethyl)-[1,3]dioxane to a mixture comprising magnesium, alkylmagnesium halide, and the compound of the formula (VII-1).

15. The method of claim 14, further comprising formation of the compound of the formula (VII-1) by coupling N,O-dimethylhydroxylamine hydrochloride and a compound of the formula (VIII-1)

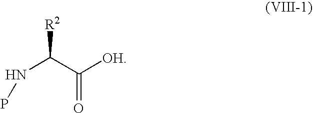

16. The method of any of claims 1-15, wherein R₂ is 3-fluoro-benzyl.

17. The method of any of claims 1-15, wherein P is carbobenzyloxy, t-butoxy carbonyl or 9-fluorenyl-methylenoxycarbonyl.

18. The method of any of claims 1-15, wherein P is t-butoxy carbonyl.

19. The method of claim 2 or 6, wherein the acid catalyst comprises ptoluenesulfonic acid, methanesulfonic acid, or sulfuric acid.

20. The method of claim 5 or 6, wherein the reducing agent comprises aluminum triisopropoxide and isopropanol.

21. The method of claim 8 or 12, wherein the reducing agent comprises N-Selectride.

22. The method of claim 8 or 12, wherein the silylating agent comprises 1,1,1,3,3,3-hexamethyl-disilazane.

23. A compound of the formula (VIg-1): 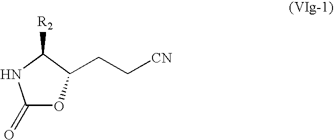
wherein:
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl(CH₂)ₘ—, (C₃-C₁₀)cycloalkyl-(CH₂)ₘ— or (C₂-C₉)heteroaryl-(CH₂)ₘ— groups may optionally be substituted with one, two, or three substituents independently selected from the group consisting of hydrogen, halogen, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-0(C═O)—, HO—(C═O)—(C₁-C₆)alkyl, (C₁-C₆)alkyl-O—(C═O)—(C₁-C₆)alkyl, (C₁-C₆)alkyl(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂NSO₂—, H₂N—SO₂—(C₁-C₆)alkyl, (C₁-C₆)alkylHN—SO₂—(C₁-C₆)alkyl, [(C₁-C₆)alkyl]₂N—SO₂(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4,
or the salts or esters thereof.

24. A compound of the formula (VIf-1): 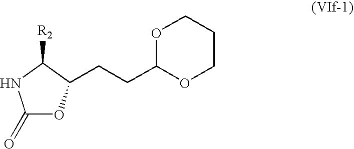
wherein:
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH-₂)ₘ—, wherein each of said phenyl, naphthyl, (C₃-C₁₀)cycloalkyl or (C₂-C₉)heteroaryl moieties of said phenyl-(CH₂)ₘ—, naphthyl(CH₂)ₘ—, (C₃-C₁₀)cycloalkyl-(CH₂)ₘ— or (C₂-C₉)heteroaryl-(CH₂)ₘ— groups may optionally be substituted with one, two, or three substituents independently selected from the group consisting of hydrogen, halogen, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-O(C═O)—, HO—(C═O)—(C₁-C₆)alkyl, (C₁-C₆)alkyl-O—(C═O)—(C₁-C₆)alkyl, (C₁-C₆)alkyl-(C═O)O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N(C═O)—, H₂N(C═O)—(C₁-C₆)alkyl, (C₁-C₆)alkyl-HN(C═O)—(C₁-C₆)alkyl, [(C₁-C₆)alkyl]₂N(C═O)—(C₁-C₆)alkyl, H(O═C)—NH—, (C₁-C₆)alkyl(C═O)—N H, (C₁-C₆)alkyl(C═O)[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)— (C₁-C₆)alkyl-SO₂—, (C₁-C₆)alkyl-SO₂—NH—, H₂N—SO₂—, H₂N—S,O₂(C₁-C₆)alkyl, (C₁-C₆)alkylHN—SO₂—, (C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4,
or the salts or esters thereof.

25. A compound of the formula (Vie-1) 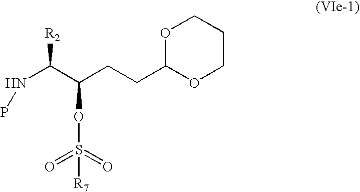
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl(CH₂)ₘ—, (C₃-C₁₀)cycloalkyl-(CH₂)ₘ— or (C₂-C₉)heteroaryl-(CH₂)ₘ— groups may optionally be substituted with one, two, or three substituents independently selected from the group consisting of hydrogen, halogen, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-O(C═O)—, HO—(C═O)—(C₁-C₆)alkyl, (C₁-C₆)alkyl-O—(C═O)—(C₁-C₆)alkyl, (C₁-C₆)alkyl(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂NSO₂—, H₂N—SO₂—(C₁-C₆)alkyl, (C₁-C₆)alkylHN—SO₂—(C₁-C₆)alkyl, [(C₁-C₆)alkyl]₂N—SO₂(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl;
R₇ is (C₁-C₆)alkyl or phenyl wherein the phenyl group may be optionally substituted with one, two, or three (C₁-C₆)alkyl, hydroxy, or halogen groups; and
m is 0, 1, 2, 3, or 4.

26. A compound of the formula (VId-1): 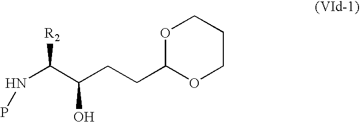
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl(CH₂)ₘ—, (C₃-C₁₀)cycloalkyl-(CH₂)ₘ— or (C₂-C₉)heteroaryl-(CH₂)ₘ— groups may optionally be substituted with one, two, or three substituents independently selected from the group consisting of hydrogen, halogen, CN, (C₁-C₆)alkyl, hydroxy, hydroxy(C₁-C₆)alkyl, (C₁—P₆)alkoxy, (C₁-C₆)alkoxy(C₁-C₆)alkyl, HO—(C═O)—, (C₁-C₆)alkyl-O(C═O)—, HO—(C═O)—(C₁-C₆)alkyl, —(C₁-C₆)alkyl-O—(C═O)—(C₁-C₆)alkyl, (C₁-C₆)alkyl(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂NSO₂—, H₂N—SO₂—(C₁-C₆)alkyl, (C₁-C₆)alkylHN—SO₂—(C₁-C₆)alkyl, [(C₁-C₆)alkyl]₂N—SO₂(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4.

27. A compound of the formula (VIb-1): 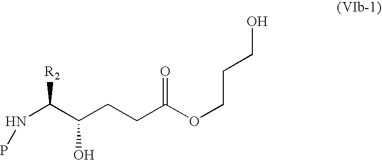
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl(CH₂)ₘ—, (C₃-C₁₀)cycloalkyl-(CH₂)ₘ— or (C₂-C₉)heteroaryl-(CH₂)ₘ— groups may optionally be substituted with one, two, or three substituents independently selected from the group consisting of hydrogen, halogen, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-O(C═O)—, HO—(C═O)—(C₁-C₆)alkyl, (C₁-C₆)alkyl-O—(C═O)—(C₁-C₆)alkyl, (C₁-C₆)alkyl(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—; (C₁-C₆)alkyl-NH—(C—O)—, [(C₁₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—N H—, (C₁-C₆)alkyl(C═O)—NH, (C₁-C₆)alkyl(C═O)-[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂NSO₂—, H₂N—SO₂—(C₁-C₆)alkyl, (C₁-C₆)alkylHN—SO₂—(C₁-C₆)alkyl, [(C₁-C₆)alkyl]₂N—SO₂(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4.

28. A compound of the formula (VIa-1) 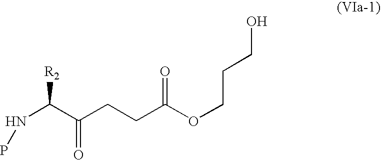
wherein:
P is a protecting group;
R₂ is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein each of said phenyl, naphthyl, (C₃-C₁₀)cycloalkyl or (C₂-C₉)heteroaryl moieties of said phenyl-(CH₂)ₘ—, naphthyl(CH₂)ₘ—, (C₃-C₁₀)cycloalkyl-(CH₂)ₘ— or (C₂-C₉)heteroaryl-(CH₂)ₘ— groups may optionally be substituted with one, two, or three substituents independently selected from the group consisting of hydrogen, halogen, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-O(C═O)—, HO—(C═O)—(C₁-C₆)alkyl, (C₁-C₆)alkyl-O—(C═O)—(C₁-C₆)alkyl, (C₁-C₆)alkyl(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂NSO₂—, H₂N—SO₂—(C₁-C₆)alkyl, (C₁-C₆)alkylH N—SO₂—(C₁-C₆)alkyl, [(C₁-C₆)alkyl]₂N—SO₂(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, and (C₂-C₉)heteroaryl; and
m is 0, 1, 2, 3, or 4.

29. A compound of the formula (VI-1): 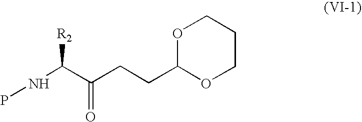
wherein P is a protecting group and R₂ is 3-fluoro-benzyl.

30. A compound of the formula (VII-2): 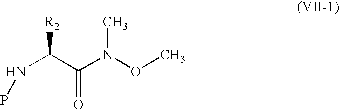
wherein P is a protecting group and R₂ is 3-fluoro-benzyl.

31. The compound of any of claims 25-30, wherein P is carbobenzyloxy, t-butoxy carbonyl or 9-fluorenyl-methylenoxy carbonyl.

32. The compound of any of claims 25-30, wherein P is t-butoxy carbonyl.

33. The compound of any of claims 23-28, wherein R₂ is 3-fluoro-benzyl.

34. The compound of any of claims 23-28, wherein P is carbobenzyloxy, t-butoxy carbonyl or 9-fluorenyl-methylenoxy carbonyl.

35. The compound of any of claims 23-28, wherein P is t-butoxy carbonyl.
